(19) United States
(12) Patent Application Publication
(10) Pub. No.: US 2023/0354935 A1
(43) Pub. Date: Nov. 9, 2023

(54) ENHANCED BIODEGRADABLE NITRILE RUBBER GLOVE AND METHODS FOR THE MANUFACTURE THEREOF

(71) Applicants: Willem Douwe Lukas DE GRAAF, Delft (NL); Daniël Nick LAMENS, Rotterdam (NL); Michiel Alphons Jacobus KUIJPERS, Den Haag (NL)

(72) Inventors: Willem Douwe Lukas DE GRAAF, Delft (NL); Daniël Nick LAMENS, Rotterdam (NL); Michiel Alphons Jacobus KUIJPERS, Den Haag (NL)

(73) Assignee: MD Biotec BV, Rotterdam (NL)

(21) Appl. No.: 17/662,427

(22) Filed: May 7, 2022

(51) Int. Cl. A41D 19/00

(52) U.S. Cl. CPC A41D 19/0055

(57) ABSTRACT

An enhanced biodegradable multilayer nitril rubber glove is disclosed, wherein the glove inner layer comprises a larger amount, more preferably a significant larger amount, of a biodegradable agent than an amount of a biodegradable agent present in the glove outer layer. After glove use such as in a hospital, disposal of the disclosed biodegradable glove is in particular intended, after shredding, for a fast thermophilic anaerobic digestion process.

### CROSS REFERENCE TO RELATED APPLICATION(S)

This Application claims priorities and subject matter disclosed in International Application Nos. PCT/NL2020/0018 filed on Nov. 3, 2020 (based on Netherland Applications No. 1043452 (NL) dated Nov. 7, 2019), and PCT/NL2020/0019 filed on Dec. 22, 2020 (based on Netherland Applications No. 1043526 (NL) dated Dec. 30, 2019), which are incorporated herein by reference in its entirety.

### FIELD OF THE INVENTION

The present invention relates generally to biodegradable gloves, and more particularly to enhanced biodegradable multilayer nitril rubber glove suitable for medical use, and methods for the manufacture thereof.

### BACKGROUND OF THE INVENTION

In the recent years, users more and more demand disposable products, such as hospital gloves, to be in a certain extent biodegradable, in order to reduce the problems the disposable waste creates for the environment. Biodegradable gloves are known in the prior art, such as disclosed in US2014/0065311, the contents of which disclosure are for ease of reading fully incorporated here within.

However, to a certain extent, the in US2014/0065311 disclosed gloves are intended for dumping on ordinary landfills, and do not match the requirements as nowadays posed by European end-users such as hospitals, since most hospital waste is not ending in a landfill but finally fed into waste incinerators. As the cost for waste incineration is growing fast, up to and above 2000 Euro per ton of waste, a need has arisen for enhanced biodegradable hospital products, in particular for enhanced biodegradable gloves.

Recently, in some hospitals the waste including solids and liquids is centrally collected and shredded in a so called Pharmafilter shredder, the liquids/solids are than separated, followed by a water treatment for the liquids and for the solids followed by an anaerobic high temperature treatment (at around 60 degrees Celcius) for a few months, after which the solids, significantly reduced in mass, are incinerated.

Another disposable glove design is disclosed in US2010/0257657 (SmartHealth), wherein polylactic acid biodegradable gloves are the subject of disclosure. This document, in disclosing only PLA based gloves, is clearly leading away from the use of nitril rubber as a base material for the gloves. However it might be of some interest for the skilled person because of the disclosed multi layered approach (see FIG. 3 in US2010/0257657, and FIG. 7 for the production method), and because of the notion that document in paragraph 29 that each layer of the polylactic acid (PLA) glove is being designed to comply with specific requirements for a given end-use application.

Yet another biodegradable multi-layer based glove is disclosed in US2013/0067635 (Inteplast Group), for ethylene based gloves only. Again this disclosure is leading away from the commonly known nitril (butyl) rubber based gloves, but this publication is still of interest for the shown multi layer approach in general. The PLA (as in US2010/0257657) and ethylene (as in US2013/0067635) based gloves are deemed inferior to nitril rubber gloves, in particular in medical environments. For those reasons a skilled person is unlikely to consider the contents of these publications when trying to further improve the biodegradability of nitril rubber based gloves.

Carboxylated nitrile rubbers can be chemically crosslinked in at least two ways: the butadiene subunits can be covalently crosslinked with sulfur/accelerator systems; and the carboxylated (organic acid) sites can be ionically crosslinked with metal oxides or salts. Sulfur crosslinks often result in large improvements in oil and chemical resistance. Ionic crosslinks, resulting from, for example, the addition of zinc oxide to the rubber, result in a rubber having high tensile strength, puncture resistance, and abrasion resistance, as well as high elastic modulus (a measure of the force required to stretch a film of the rubber), but poor oil and chemical resistance.

Many currently available rubber formulations generally employ a combination of the two curing mechanisms. For example, in combination with sulfur and accelerators, carboxylated nitrile rubber manufacturers frequently recommend addition of 1-10 parts of zinc oxide per 100 parts of rubber (as is also disclosed in par. 129 and 130 in US2014/0065311.

When zinc oxide is not employed, the curing time required to reach an optimum state of cure can be much longer and the curing may be less efficient. This means that the crosslinks are longer (more sulfur atoms per crosslink) and there may be a higher amount of sulfur that does not crosslink polymer chains. The result can be a less-effectively cured rubber that has lowered heat resistance and less chemical resistance.

However, ionic crosslinking often increases the stiffness of an article made from the rubber. This is a disadvantage for applications in which a softer rubber is needed. For example, surgical gloves made of soft rubbers can provide greater tactile sensitivity for the wearer, which is desirable to improve the surgeon's “feel” during operations and to prevent fatigue of the hands.

A more comfortable nitrite glove that is easier to stretch, i.e. has lower elastic modulus, can be made using a polymer which contains less acrylonitrile or by crosslinking the polymer to a lesser degree. These changes, however, often compromise strength, chemical resistance, or both, resulting in articles that are unsuitable for many applications. Accordingly, a soft rubber having strength and chemical resistance similar to stiffer rubbers is highly desirable.

U.S. Pat. No. 2,868,754 discloses the formation and use of carboxyl-containing elastomers. The problems associated within the use of zinc oxide curing agents are overcome in this document by using alkali metal aluminates. Another method includes the steps of combining a nitrile latex base with a stabilizing agent, such as ammonium caseinate, and adjusting the pH of the nitrile latex to about 8.5-10.0 to yield a basic nitrile rubber. The basic nitrile rubber is contacted with a substantially metallic-oxide-free sulphur crosslinking agent and with at least one accelerator, to form a nitrile rubber composition. That composition is substantially free of metallic oxide.

Another known biodegradable nitril rubber glove is known under the brand name Powerform S6 Ecotek. These gloves use a single layered glove design made of nitril rubber mixed with a naturally occurring microorganism, which makes them suitable for industrial applications.

One latest relevant disclosures on biodegradable elastomeric compositions, including nitril rubber, is given in WO2019/074354. Therein stated disclosure for elastomers in general does form some relevant prior art but also indicates the rather large step the present inventors had to take in order to arrive at the nitril rubber gloves forming the subject matter as disclosed in the present invention.

The objective of this invention is to prepare nitril rubber based gloves, such as for example disclosed in US2014/0065311, in particular also including the soft nitril rubber gloves such as those disclosed in EP0925329, with a strongly enhanced biodegradable functionality, complying to all up to date generally known norms and standards, such as the norms and standards listed in US2014/0065311, for hospital or medical examination gloves such as for strength and chemical resistance, but as well as for the latest 2019 biodegradation norms and standards.

For this application, more focus will further be placed on the so called double dipped gloves, such as disclosed in US2007/0033704 (Encompass Medical Supplies), wherein a method of making a glove is described dipping a glove form into two different formulations, with different amounts of covalent and ionic cross linkers. In US2007/0033704 no link whatsoever is made to obtaining a biodegradable NBR nitril rubber glove, but after extensive testing applicant has found that in particular this double dipping method of making a glove, when adapted and modified, is in particular suited for obtaining a biodegradable nitril rubber glove with acceptable and comfortable stress retention features.

Furthermore, as the trend is towards thinner nitril rubber gloves, for less cost and less pollution, it has been found by surprise that the double dipped versions of biodegradable nitril rubber gloves perform better as initially expected in the mechanical puncture tests, even when loaded with significant biodegradable agent and/or biobased filler amounts which is required in order to get acceptable biodegradability results. Preferably the maximum possible bio-based filler addition that can be added while the nitrile rubber compound remains the matrix phase. NBR nitril rubber is incomparable to, for example, PLA based materials, and requires a totally novel approach concerning the additives loading.

In a most recent publication published in Applied Chemistry and Biotechnology, “Unlocking the secret of bio-additive components in rubber compounding in processing quality nitrile glove” by Guo Yong Yew et. al., https://doi.org/10.1007/s12010-019-033207-7, received 26 Aug. 2019 and published online 31 Jan. 2020 @ Springer Science+Business Media, a detailed overview of some possible bio-additives with biodegradable properties falling within the scope of this application is supplied. For the sake of clarity in this application a broad definition of biodegradable property is adhered to, some examples hereunder are defined in more detail for a further explaining the broad concept of this invention. In addition to the bio-additives also bio-based substances can be mixed into the bio-additives with biodegradable properties, such an overall mixture is also to be regarded as a biodegradable agent in the light of this application.

Starch and/or alginate (and/or natriumalginate, in which an alginate is defined as to comprise a polysaccharide derived from for example brown seaweed known as Phaeophyceae) are candidates for a further improvement of the bio-based additives, in order to reduce the overall environmental waste load. In combination with a carboxylated nitril rubber (also known under the general acronym XNBR) natural organics such as starch and alginate can form another network like structural enhancement after curing the glove, and combinations of nitril rubber, in particular of a base material made of mainly XNBR (more than 40% XNBR based on weight), comprising these bio-based fillers are within the scope as defined by the claims. Preferably the maximum possible bio-based filler addition that can be added while the nitrile rubber compound remains the matrix phase.

Accordingly, there exists a need for environment friendly, convenient, secure and coast effective enhanced biodegradable multilayer nitril rubber glove suitable for medical use.

### SUMMARY OF THE INVENTION

In view of the foregoing shortcomings inherent in the conventional globes, the general purpose of the present invention is to provide an enhanced biodegradable multilayer nitril rubber glove suitable for medical use, and methods for the manufacture thereof, to include the advantages of the prior art, and to overcome the drawbacks inherent therein.

In one aspect, the present invention provides an enhanced biodegradable multilayer nitril rubber glove suitable for medical use, comprising: an inner layer intended for contacting a hand; and an outer layer suitable for contacting objects; wherein the inner layer comprises a relative larger amount of a biodegradable agent than a relative amount of a biodegradable agent present in the outer layer.

In another aspect, the present invention provides an enhanced biodegradable multilayer nitril rubber glove suitable for medical use, comprising: an inner layer intended for contacting a hand; an outer layer suitable for contacting objects; wherein a nitril compound part comprises at least one of a pure NBR, a pure XNBR, a NBR, a XNBR compound or any combination thereof mixed with up to one of 20% weight percentage and 40% weight percentage of a biobased filler such as a starch, an alginate, wherein the outer layer comprises a larger amount of a biodegradable agent than an amount of a biodegradable agent present in the inner layer.

In another aspect, the present invention provides a method of producing an enhanced biodegradable nitril rubber glove, comprising steps of: forming around a glove former a first inner layer of a nitril rubber having a relative amount of biodegradable agent which is one of larger than 2 parts per 100 dry parts nitril rubber, larger than 3 parts per 100 dry parts nitril rubber, larger than 4 parts per 100 dry parts nitril rubber; and forming of an outer layer of a nitril rubber having a relative amount of biodegradable agent which is one of lower than or equal to 2 parts per 100 dry parts nitril rubber or between 1 and 2 parts per 100 dry parts nitril rubber.

In another aspect, the present invention provides a method of producing an enhanced biodegradable nitril rubber glove, comprising the steps of: forming around a glove former a first inner layer of a nitril rubber, wherein the nitril compound part comprise one of a pure NBR, a pure XNBR, a NBR, a XNBR compound or any combination thereof mixed with up to one of 20% weight percentage, 40% weight percentage, the first inner layer further comprising an amount of a biodegradable agent with one of proportions lower than or equal to 2 parts per 100 dry parts nitril rubber, between 1 and 2 parts per 100 dry parts nitril rubber; forming of an outer layer of a nitril rubber, wherein a nitril compound part comprise one of a pure NBR, a pure XNBR, a NBR, XNBR compound mixed with up to one of 20% weight percentage, 40% weight percentage, the outer layer further comprising an amount of biodegradable agent larger than one of 2 parts per 100 dry parts nitril rubber, 3 parts per 100 dry parts nitril rubber, 4 parts per 100 dry parts nitril rubber.

In another aspect of the present invention, after the forming of the first layer, and before the forming of the outer layer, an additional intermediate nitril rubber layer is formed on the glove former, the additional intermediate layer also comprising at least one of a biodegradable agent and a biobased filler material mixed inside the nitril rubber composition.

These together with other aspects of the present invention and various features of novelty that characterize the invention, are pointed out particularity in the detailed description forming a part of this disclosure.

### DETAILED DESCRIPTION OF THE INVENTION

It is to be understood, that the foregoing general and following detailed description are exemplary and explanatory only and are in no way not restrictive to the invention, as defined in the claims.

The definitions and all technical and scientific terms in the following description are identical as to those defined in US2014/0065311, this applies to terms such as for example “biodegradable”, “ranges”, “polymer”, “glove former” etc. etc.

The term nitrile rubber in this application also includes so called soft nitrile rubber formulations, such as disclosed in EP0925329, wherein it is explained in more detail how crosslinking increases the strength and elasticity of the rubber.

According an aspect of the present invention, an enhanced biodegradable multilayer nitril rubber glove suitable for medical use, comprising: an inner layer intended for contacting a hand; and an outer layer suitable for contacting objects; wherein the inner layer comprises a relative larger amount of a biodegradable agent than a relative amount of a biodegradable agent present in the outer layer. The inner layer is thicker as the outer layer, preferably the inner layer is at least 50% thicker as the outer layer. The glove material comprises one of at least 50% nitril rubber, at least 70% nitril rubber, at least 90% nitril rubber, all based on dry parts nitril rubber. The relative amount of the biodegradable agent in the inner layer is one of larger than 2 parts per 100 dry parts nitril rubber, larger than 3 parts per 100 dry parts nitril rubber, larger than 4 parts per 100 dry parts nitril rubber. The relative amount of the biodegradable agent in the outer layer is lower than or equal to 2 parts per 100 dry parts nitril rubber or between 1 and 2 parts per 100 dry parts nitril rubber. The nitril rubber is a soft cured nitril rubber comprising a sulfur based crosslinking agent. Biodegradation agents are adapted and optimized for enhanced glove biodegradation in a heated anaerobic digester, wherein the digester working preferably at or around 60 degrees Celsius. The inner layer and the outer layer are separated by an additional nitril rubber layer, the additional layer further comprising at least one of the biodegradable agent and a biobased filler. A proportion of a relative amount of the biodegradable agent in weight % in the inner layer to a relative amount of the biodegradable agent in weight % in the outer layer is one of greater than 1.0, greater than 1.4, greater than 2.0. The biodegradable agent or an agent mixture is preferably adapted for a thermophilic digestion biodegradation processor or to an anaerobic digestion at elevated temperatures above 60 degree Celsius during a time span of more than 30 days.

According to another aspect of the present invention, an enhanced biodegradable multilayer nitril rubber glove suitable for medical use, comprising: an inner layer intended for contacting a hand; an outer layer suitable for contacting objects; wherein a nitril compound part comprises at least one of a pure NBR, a pure XNBR, a NBR, a XNBR compound or any combination thereof mixed with up to one of 20% weight percentage and 40% weight percentage of a biobased filler such as a starch, an alginate, wherein the outer layer comprises a larger amount of a biodegradable agent than an amount of a biodegradable agent present in the inner layer. The outer layer is thicker as the inner layer, preferably the outer layer is at least 50% thicker as the inner layer. The amount of the biodegradable agent in the outer layer is one of larger than 2 parts per 100 dry parts nitril rubber, larger than 3 parts per 100 dry parts nitril rubber, larger than 4 parts per 100 dry parts nitril rubber. The amount of the biodegradable agent in the inner layer is lower than or equal to 2 parts per 100 dry parts nitril rubber or between 1 and 2 parts per 100 dry parts nitril rubber

According to another aspect of the present invention, a method of producing an enhanced biodegradable nitril rubber glove, comprising steps of: forming around a glove former a first inner layer of a nitril rubber having a relative amount of biodegradable agent which is one of larger than 2 parts per 100 dry parts nitril rubber, larger than 3 parts per 100 dry parts nitril rubber, larger than 4 parts per 100 dry parts nitril rubber; and forming of an outer layer of a nitril rubber having a relative amount of biodegradable agent which is one of lower than or equal to 2 parts per 100 dry parts nitril rubber or between 1 and 2 parts per 100 dry parts nitril rubber.

According to another aspect of the present invention, a method of producing an enhanced biodegradable nitril rubber glove, comprising the steps of: forming around a glove former a first inner layer of a nitril rubber, wherein the nitril compound part comprise one of a pure NBR, a pure XNBR, a NBR, a XNBR compound or any combination thereof mixed with up to one of 20% weight percentage, 40% weight percentage, the first inner layer further comprising an amount of a biodegradable agent with one of proportions lower than or equal to 2 parts per 100 dry parts nitril rubber, between 1 and 2 parts per 100 dry parts nitril rubber; forming of an outer layer of a nitril rubber, wherein a nitril compound part comprise one of a pure NBR, a pure XNBR, a NBR, XNBR compound mixed with up to one of 20% weight percentage, 40% weight percentage, the outer layer further comprising an amount of biodegradable agent larger than one of 2 parts per 100 dry parts nitril rubber, 3 parts per 100 dry parts nitril rubber, 4 parts per 100 dry parts nitril rubber.

In another aspect of the present invention, after the forming of the first layer, and before the forming of the outer layer, an additional intermediate nitril rubber layer is formed on the glove former, the additional intermediate layer also comprising at least one of a biodegradable agent and a biobased filler material mixed inside the nitril rubber composition.

According yet another aspect of the present invention, a method of biodegrading the nitril rubber glove comprising the steps of: shredding the nitril rubber globe after use into smaller particles; and digesting the shredded small particles of the rubber globe by an anaerobic digestion in a digester at elevated temperatures, during a period of in between 20 to 200 days in combination with an addition of further shredded medical or hospital waste such as bandages, clothes, linings, wherein during the anaerobic digestion additional biodegrading enhancing supplements or agents are added into the digester in order to enhance the further acceleration of the nitril rubber degradation.

After own extensive testing of the biodegradable gloves produced following the US2014/0065311 disclosure, it appeared most functionalities could be met within norms and standards, however in particular not for the aspect that these gloves shall degrade much faster as is required by modern hospital use. A normal solution would be, to add a bit more biodegrading agent, however was shown only possible to a very limited extent for strength and other required functional requirements.

Knowing the double dipped nitril rubber gloves disclosed in US2007/0033704, testing started for how to develop a more enhanced, more biodegradable nitril rubber glove, using the combined know how supplied in the documents US2014/0065311 and US2007/0033704.

While on a normal landfill the outside of the glove remains outside forever, the difference with the recent practice in especially designed hospital waste treatment systems (based on for example EP2859952 and EP3015750 Pharmafilter B.V.) can be used to a common advantage to create an improved biodegradability of the nitril rubber glove.

Because in modern hospital waste disposal systems a shredder will shred the complete glove in pieces, with these pieces sized in the cm range (so pieces with dimensions of around 0.5 to 5 cm large), an inside and outside of the glove will not exist anymore once anaerobic digestion (storage) at around 60 degrees Celsius begins to activate the biodegradable agent and the NBR nitril rubber starts to degrade, under emission of gasses sucked away (which might be used as biogas for heating purposes)

By adding relatively much more biodegrading agent into a glove inner layer, where initially it does not have too much a detrimental effects on the glove functional requirements, and keeping the outer layer filled with less biodegrading agent as an additive, overall glove biodegradation is significantly enhanced under modern hospital waste treatment condition, while keeping a fully functional nitril rubber glove, displaying an uncompromised quality performance when in use. By adding relatively much more biodegrading agent into a glove outer layer, where initially it does not have too much a detrimental effects on the glove functional requirements (for example, sharp nails on the inside), and keeping the inner layer filled with less biodegrading agent as an additive, overall glove biodegradation is significantly enhanced under modern hospital waste treatment condition, while keeping a fully functional nitril rubber glove, displaying an uncompromised quality performance when in use. By creating two different layers, each having significantly different biodegradation agent, a stronger double dipped nitril rubber glove can be created which is performing well in biodegrading after use.

Against common wisdom, this invention adds much more biodegradable agent to the nitril rubber polymer as is conventionally known and disclosed, such as in US2014/0065311 (Showa), wherein 2% of a biodegradable agent as additive is clearly regarded as upper limit. The present invention provides a double dipped enhanced biodegradable nitrile rubber glove. By additionally applying a double dipping technique two mechanically slightly differently behaving layers are created which has a surprising positive effect on in particular the needle puncture resistance, leading to an acceptable mechanical performance as required by the standards (such as the ASTM), but with a significantly higher biodegradable agent load creating a much better biodegradability. As the known biodegrading agents and the known biobased fillers are not so costly, in practice the additional cost for the end customer can be limited to amounts of a maximum of 2 dollar per 1000 gloves, this is deemed to be still acceptable for a requirement expected to be legally mandatory in the not so far future.

Example: A nitril rubber glove has been prepared, wherein first an inner nitril rubber layer comprising 5% (by weight) or 2% (by weight, which is considered very high by the skilled person) of a biodegradable agent (in this case: ECM Biofilms Masterbatch pellets, comprising Low Density Polyethylene pellets with additive ingredients of Organoleptic-Organic chemical names/cultured colloids, Harmonized system based Schedule B code ECM6.0404 is 3901.10.5020) is first produced around a glove former, followed by a second outer layer on the glove former applying a nitril rubber comprising only 2% (by dry parts) or 5% (by dry parts, so significantly more than in the inner layer) of the same biodegradable agent composition.

The effect on the biodegradation, as per ASTM D5511 “Standard test method for determining anaerobic biodegradation of plastic materials under high-solids anaerobic digestion conditions”, of an, as in example 1, produced glove has been tested in a real life hospital environment, using a “Pharmafilter B.V.” shredding first, followed by a 6 week anaerobic digester storage at 60 degrees Celsius. The effects were surprisingly convincing, as was furthermore proven by a more than 80% or 100% larger CO2 production in the resulting off-gassing.

Biodegradability in the ASTM D5511 test came at least 25% or 40% higher as compared to the percentage biodegradation of the corresponding reference material (using for example gloves known in the market place as Showa Best Gloves with Eco Best Technology, apparently based on US2014/0065311 technology). But what was even more important was the strongly improved digester biodegradation at temperatures of 60 degrees Celsius, after 60 days (compared to the Showa Best Gloves) an improval rate of over 75% or 100% in biodegradation effect was measured.

Wherein after 60 days in the digester (after shredding) the Showa Best gloves with Eco Best Technology showed a biodegradation in the range of 4 to 5%, the gloves according to the invention were at least 6 to 9% and 9 to 10% biodegraded, with potentially a much further degradation after 300 or 900 days in the range of 30 up to 85 or 90% overall biodegradation.

Other components such as a swelling agents, or other chemo attractant agents, can/may be added in small amounts for enhancing the biodegradation effects, but were regarded not yet necessary for this comparative example. Other chemistries, such as alkali stabilizers, as indicated in US2014/0065311, as included in its entirety for reference again, can be included in minor quantities in any of the glove layers for the usual and obvious functional reasons (this also includes, if required, curing agents). Any of the biodegradable components mentioned in US2014/0065311 can be mixed into another blend of a suitable biodegradable agent, and mixed into one the glove layers as per claim 1 and any of the dependent claims.

From document US2014/0065311, for being clear about what is regarded a biodegradable agent as defined in this application, a non-exhaustive listing of possible biodegradable agents is hereby listed, suitable to be used as components in order to enhance the degradation of the nitril rubber glove after use: Biodegradation is generally considered as consisting of either enzyme-catalyzed hydrolysis, non-enzymatic hydrolysis, metabolic action, or both. The enzymes may be either endoenzymes which cleave the internal chain linkages within the chain or exoenzymes which cleave terminal monomer units sequentially.

Biodegradation is a functional decay of material, e.g. loss of strength, substance, transparency, or good dielectric properties where it is known to be identifiable with exposure of the material to a living environment, which may itself be very complex, and the property loss may be attributable to physical or chemical actions as first steps in an elaborate chain of processes.

A biodegradable polymer is a high molecular weight polymer that, owing to the action of micro- and/or macroorganisms or enzymes, degrades to lower molecular weight compounds. Natural polymers are by definition those which are biosynthesized by various routes in the biosphere. Proteins, polysaccharides, nucleic acids, lipids, natural rubber, and lignin, among others, are all biodegradable polymers, but the rate of this biodegradation may vary from hours to years depending on the nature of the functional group and degree of complexity. Biopolymers are organized in different ways at different scales. This hierarchical architecture of natural polymers allows the use of relatively few starting molecules (i.e. monomers), which are varied in sequences and conformations at molecular-, nano-, micro-, and macroscale, forming truly environmentally adaptable polymers.

On the other hand, the repetitive units of synthetic polymers are hydrolyzable, oxidizable, thermally degradable, or degradable by other means. Nature also uses these degradation modes, e.g., oxidation or hydrolysis, so in that sense there is no distinction between natural or synthetic polymers. The catalysts promoting the degradations in nature (catabolisms) are the enzymes, which are grouped in six different classes according to the reaction catalyzed. These classes include oxidoreductase for catalyzing redox reactions, transferase for catalyzing transfer of functional group reactions, hydrolase for catalyzing hydrolysis, lyase for catalyzing addition to double bond reactions, isomerase for catalyzing isomerization and ligase for catalyzing formation of new bonds using ATP.

Biodegradation of oxidizable polymers is generally slower than biodegradation of hydrolyzable ones. Even polyethylene, which is rather inert to direct biodegradation, has been shown to biodegrade after initial photo-oxidation. An oxidized polymer is more brittle and hydrophilic than a non-oxidized polymer, which also usually results in a material with increased biodegradability.

For example, by combining a nickel dithiocarbamate (photo antioxidant) with an iron dithiocarbamate (photo proxidant), a wide range of embrittlement times may be obtained.

The gloves disclosed herein provides for increased susceptibility to biodegradation of nitril rubber by means of additives including a biopolymer. In this way a nitril rubber polymer blend is obtained that is more susceptible to biodegradation.

A filler might be added to a composition to be added to a polymer thereby increasing the biodegradability. For relevant fillers, reference is also made to claims as stated in WO2019/074354. Starch is of course one of the more popular fillers (as in Starch-graft-acrylonitrile ANS), however also collagen is a known filler in combination with nitril rubber, as well as is keratine. Another bio-based known filler can be alginate (or natriumalginate) which has some surprising effects in combination with XNBR as a base material, especially for a douple dipped based glove.

Microbial or enzymatic attack of pure aromatic polyester is increased by exposure to certain microbes, for example Trichosporum, athrobacteria and Asperyillus negs.

Aliphatic polyester degradation is seen as a two-step process: the first is depolymerization, or surface erosion. The second is enzymatic hydrolysis which produces water-insoluble intermediates that can be assimilated by microbial cells.

Polyurethane degradation may occur by fungal degradation, bacterial degradation and degradation by polyurethane enzymes.

In one aspect, the biodegradation agent can be a polymer, such as a biodegradable polymer. The polymers can be homo- or co-polymers. In one aspect, the polymer is a homopolymer. In another aspect, the polymer is a co-polymer. Co-polymers include AB and ABA type co-polymers. In one aspect, the polymer comprises polylactic acid, poly(lactic-co-glycolic acid), polypolypropylene carbonate, polycaprolactone, polyhydroxyalkanoate, chitosan, gluten, and one or more aliphatic/aromatic polyesters such as polybutylene succinate, polybutylene succinate-adipate, polybutylene succinate-sebacate, or polybutylene terephthalate-coadipate, or a mixture thereof.

In one aspect the polymer is polybutylene succinate. In one aspect, the polybutylene succinate can have a number average molecular mass (Mn) from 1,000 g/mole to 100,000 g/mole.

The biodegradation agent may comprise a carboxylic acid compound. The biodegradation agent may comprise a chemo attractant compound; a glutaric acid or its derivative; a carboxylic acid compound with chain length from 5-18 carbons; a polymer; and a swelling agent.

The biodegradation agent may further comprise a microbe capable of digesting the acrylonitrile butadiene based (nitril) rubber.

The polymer that may be comprised in the biodegradation agent can be selected from the group consisting of: polydivinyl benzene, ethylene vinyl acetate copolymers, polyethylene, polypropylene, polystyrene, polyterephthalate, polyesters, polyvinyl chloride, methacrylate, nylon 6, polycarbonate, polyamide, polychloroprene, acrylonitrile butadiene based rubber, and any copolymers of said polymers, or a combination thereof.

The biodegradation agent may further comprise a compatibilizing additive.

The biodegradation agent may further comprise a carrier resin. Suitable carrier resins include, but are not limited to, polydivinyl benzene, ethylene vinyl acetate copolymers, maleic anhydride, and acrylic acid with polyolefins, or a combination thereof.

The biodegradation agent may further comprise a chemotaxis agent to attract microbes. Suitable chemotaxis agents comprise, but are not limited to, a sugar or a furanone. It can be selected from 3, 5 dimethylyentenyl dihydro 2(3H)furanone isomer mixtures, emoxyfurane and N-acylhomoserine lactones. The chemotaxis agent may comprise coumarin and/or coumarin derivatives.

Without being bound by theory, it is believed that the biodegradation agent enhances the biodegradability of otherwise non-biodegradable plastic products through a series of chemical and biological processes when disposed of in a microbe-rich environment, such as a biologically active landfill or a digester tank held at elevated temperatures in between 50 to 70 deg C. The biodegradation agent causes the plastic to be an attractive food source to certain soil microbes, encouraging the plastic to be consumed more quickly than plastics without the biodegradation agent.

The biodegradation agent requires the action of certain enzymes for the biodegradation process to begin, so plastics containing the biodegradation agent will not start to biodegrade during the intended use of the materials and gloves described herein. For example, the microbes can secrete enzymes that break down the polymers into components that are easily consumed by microbes. Typically, when an organic material biodegrades in an anaerobic environment, the by-products are: humus, methane and carbon dioxide. It is believed that when plastics containing the biodegradation agent are biodegraded to the same by-products as an organic material.

Biodegradation processes can affect polymers in a number of ways. Microbial processes that can affect polymers include mechanical damage caused by growing cells, direct enzymatic effects leading to breakdown of the polymer structure, and secondary biochemical effects caused by excretion of substances other than enzymes that may directly affect the polymer or change environmental conditions, such as pH or redox conditions. Although microorganisms such as bacteria generally are very specific with respect to the substrate utilized for growth, many are capable of adapting to other substrates over time. Microorganisms produce enzymes that catalyze reactions by combining with a specific substrate or combination of substrates. The conformation of these enzymes determines their catalytic reactivity towards polymers. Conformational changes in these enzymes may be induced by the changes in pH, temperature, and other chemical additives.

Microbes that may assist in biodegradation are psychrophiles, mesophiles, thermophiles, actinomycetes, saprophytes, absidia, acremonium, alternaria, amerospore, arthrinium, ascospore, aspergillus, Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus ochraceus, Aspergillus oryzae, Aspergillus parasiticus, Aspergillus penicilloides, Aspergillus restrictus, Aspergillus sydowii, Aspergillus terreus, Aspergillus ustus, Aspergillus versicolor, Aspergillus/penicillium-like, aureobasidium, basidiomycetes, basidiospore, bipolaris, blastomyces, B. borstelensis, botrytis, candida, cephalosporium, chaetomium, cladosporium, Cladosporium fulvum, Cladosporium herbarum, Cladosporium macrocarpum, Cladosporium sphaerospermum, conidia, conidium, conidobolus, Cryptococcus neoformans, cryptostroma corticale, cunninghamella, curvularia, dreschlera, epicoccum, epidermophyton, fungus, fusarium, Fusarium solani, geotrichum, gliocladium, helicomyces, helminthosporium, histoplasma, humicula, hyaline mycelia, memnoniella, microsporum, mold, monilia, mucor, mycelium, myxomycetes, nigrospora, oidium, paecilomyces, papulospora, penicillium, periconia, perithecium, peronospora, phaeohyphomycosis, phoma, pithomyces, rhizomucor, rhizopus, rhodococcus, rhodotorula, rusts, saccharomyces, scopulariopsis, sepedonium, serpula lacrymans, smuts, spegazzinia, spore, sporoschisma, sporothrix, sporotrichum, stachybotrys, stemphylium, syncephalastrum, Thermononespore fusca DSM43793, torula, trichocladium, trichoderma, trichophyton, trichothecium, tritirachium, ulocladium, verticillium, wallemia and yeast.

One or several furanone compounds combined can act as chemo attractants for bacteria and or as odorants for the decomposing or degrading polymer. Some furanones, particularly certain halogenated furanones are quorum sensing inhibitors. Quorum sensing inhibitors are typically low-molecular-mass molecules that cause significant reduction in quorum sensing microbes. In other words, halogenated furanones kill certain microbes. Halogenated furanones prevent bacterial colonization in bacteria such as V. fischeri, Vibrio harveyi, Serratia ficaria and other bacteria. However, the natural furanones are ineffective against P. aeruginosa, but synthetic furanones can be effective against P. aeruginosa.

Some furanones, including but not limited to those listed below, can be chemo attractant agents for bacteria. Suitable furanones include but are not limited to: 3,5 dimethylyentenyl dihydro 2(3H)furanone isomer mixtures, emoxyfurane and N-acylhomoserine lactones, or a combination thereof.

Bacteria that have shown to attract to the furanone compounds listed above include, but are not limited to C. violaceum.

Other chemo attractant agents include sugars that are not metabolized by the bacteria. Examples of these chemo attractant agents may include but are not limited to: galactose, galactonate, glucose, succinate, malate, aspartate, serine, fumarate, ribose, pyruvate, oxalacetate and other L-sugar structures and D-sugar structures but not limited thereto. Examples of bacteria attracted to these sugars include, but are not limited to Escherichia coli, and Salmonella. In a preferred embodiment the sugar is a non-estererfied starch.

The biodegradation agents (such as delivered under the Brand names of Enso Plastics) are combined with an acrylonitrile butadiene based rubber. When combined in small quantities with any of acrylonitrile butadiene based rubber, the resulting gloves become biodegradable while maintaining their desired characteristics. The resulting materials and products (i.e. gloves) made therefrom exhibit the same desired mechanical properties, and have effectively similar shelf-lives as products without the additive, and yet, when disposed of, are able to at least partially metabolize into inert biomass by the communities of anaerobic and aerobic microorganisms commonly found almost everywhere on Earth.

This biodegradation process can take place aerobically or anerobically. It can take place with or without the presence of light. Traditional polymers and products therefrom are now able to biodegrade in land fill and compost environments within a reasonable amount of time as defined by the EPA to be 30 to 50 years on average.

The biodegradation agents increase, when added, the biodegradation rate of the disclosed gloves. The gloves can be degraded into an inert humus-like form that is harmless to the environment. An example of attracting microorganisms through chemotaxis is to use a positive chemotaxis, such as a scented polyethylene terephthalate pellet, starch D-sugars not metabolized by the microbes or furanone that attracts microbes or any combination thereof.

The biodegradation process might begin with one or more proprietary swelling agents that, when combined with heat and moisture, expands the plastics' molecular structure. After the one or more swelling agents create space within the plastic's molecular structure, the combination of bio-active compounds discovered after significant laboratory trials attracts a colony of microorganisms that break down the chemical bonds and metabolize the plastic through natural microbial processes.

The biodegradation agent might comprise a furanone compound, a glutaric acid, a hexadecanoic acid compound, a polycaprolactone polymer, a carrier resin to assist with placing the additive material into the polymeric material in an even fashion to assure proper biodegradation. The biodegradation agent can also comprise organoleptic organic chemicals as swelling agents i.e. natural fibers, cultured colloids, cyclo-dextrin, polylactic acid, etc.

The carrier resin may be selected from, but is not limited to the group of: ethylene vinyl acetate, poly vinyl acetate, maleic anhydride, and acrylic acid with polyolefins. The biodegradation agent may further comprise dipropylene glycol.

The biodegradation agent may be incorporated in the polymers described herein by, for example, granulation, powdering, making an emulsion, suspension, or other medium of similar even consistency. The biodegradation agent may be blended into the polymeric material just before sending the nitril rubber material to the forming machinery for making the gloves.

Any carrier resin may be used (such as poly-vinyl acetate, ethyl vinyl acetate, etc.) where poly olefins or any plastic material that these carrier resins are compatible with can be combined chemically and allow for the dispersion of the additive.

The biodegradation agent may comprise one or more antioxidants that are used to control the biodegradation rate. Antioxidants can be enzymatically coupled to biodegradable monomers such that the resulting biodegradable polymer retains antioxidant function. Antioxidant-couple biodegradable polymers can be produced to result in the antioxidant coupled polymer degrading at a rate consistent with an effective administration rate of the antioxidant. Antioxidants are chosen based upon the specific application, and the biodegradable monomers may be either synthetic or natural.

An exemplary biodegradation agent may comprise the organic lipid based SR5300 product available from ENSO Plastics of Mesa, Ariz. An exemplary nitril rubber may comprise as a starting material the XNBR based Syntomer 6338 product, especially intended for ultra thin gloves.

Those skilled in the art will recognize, or be able to ascertain using no more than routine experimentation, numerous equivalents to the specific gloves described herein. Such equivalents are considered to be within the scope of this invention and are covered by the claims.

What is claimed is:

1. An enhanced biodegradable multilayer nitril rubber glove suitable for medical use, comprising:
an inner layer intended for contacting a hand; and
an outer layer suitable for contacting objects;
wherein the inner layer comprises a relative larger amount of a biodegradable agent than a relative amount of a biodegradable agent present in the outer layer.

2. The glove according to claim 1, wherein the inner layer is thicker as the outer layer, preferably the inner layer is at least 50% thicker as the outer layer.

3. The glove according to claim 1, wherein the glove material comprises one of at least 50% nitril rubber, at least 70% nitril rubber, at least 90% nitril rubber, all based on dry parts nitril rubber.

4. The glove according to claim 1, wherein the relative amount of the biodegradable agent in the inner layer is one of larger than 2 parts per 100 dry parts nitril rubber, larger than 3 parts per 100 dry parts nitril rubber, larger than 4 parts per 100 dry parts nitril rubber.

5. The glove according to claim 1, wherein the relative amount of the biodegradable agent in the outer layer is lower than or equal to 2 parts per 100 dry parts nitril rubber or between 1 and 2 parts per 100 dry parts nitril rubber.

6. The glove according to claim 1, wherein the nitril rubber is a soft cured nitril rubber comprising a sulfur based crosslinking agent.

7. The glove according to claim 1, wherein biodegradation agents are adapted and optimized for enhanced glove biodegradation in a heated anaerobic digester, wherein the digester working preferably at or around 60 degrees Celsius.

8. The glove according to claim 1, wherein the inner layer and the outer layer are separated by an additional nitril rubber layer, the additional layer further comprising at least one of the biodegradable agent and a biobased filler.

9. The glove according to claim 1, wherein a proportion of a relative amount of the biodegradable agent in weight % in the inner layer to a relative amount of the biodegradable agent in weight % in the outer layer is one of greater than 1.0, greater than 1.4, greater than 2.0.

10. The glove according to claim 1, wherein the biodegradable agent or an agent mixture is preferably adapted for a thermophilic digestion biodegradation processor or to an anaerobic digestion at elevated temperatures above 60 degree Celsius during a time span of more than 30 days.

11. An enhanced biodegradable multilayer nitril rubber glove suitable for medical use, comprising:
an inner layer intended for contacting a hand;
an outer layer suitable for contacting objects;
wherein a nitril compound part comprises at least one of a pure NBR, a pure XNBR, a NBR, a XNBR compound or any combination thereof mixed with up to one of 20% weight percentage and 40% weight percentage of a biobased filler such as a starch, an alginate,
wherein the outer layer comprises a larger amount of a biodegradable agent than an amount of a biodegradable agent present in the inner layer.

12. The glove according to claim 11, wherein the outer layer is thicker as the inner layer, preferably the outer layer is at least 50% thicker as the inner layer.

13. The glove according to claim 11, wherein the amount of the biodegradable agent in the outer layer is one of larger than 2 parts per 100 dry parts nitril rubber, larger than 3 parts per 100 dry parts nitril rubber, larger than 4 parts per 100 dry parts nitril rubber.

14. The glove according to claim 11, wherein the amount of the biodegradable agent in the inner layer is lower than or equal to 2 parts per 100 dry parts nitril rubber or between 1 and 2 parts per 100 dry parts nitril rubber.

15. A method of producing an enhanced biodegradable nitril rubber glove, comprising steps of:
forming around a glove former a first inner layer of a nitril rubber having a relative amount of biodegradable agent which is one of larger than 2 parts per 100 dry parts nitril rubber, larger than 3 parts per 100 dry parts nitril rubber, larger than 4 parts per 100 dry parts nitril rubber; and
forming of an outer layer of a nitril rubber having a relative amount of biodegradable agent which is one of lower than or equal to 2 parts per 100 dry parts nitril rubber or between 1 and 2 parts per 100 dry parts nitril rubber.

16. The method as according to claim 15, wherein after the forming of the first layer, and before the forming of the outer layer, an additional intermediate nitril rubber layer is formed on the glove former, the additional intermediate layer also comprising at least one of a biodegradable agent and a biobased filler material mixed inside the nitril rubber composition.

17. The method according to claim 15, wherein a method of biodegrading the nitril rubber glove comprising the steps of:
shredding the nitril rubber globe after use into smaller particles; and
digesting the shredded small particles of the rubber globe by an anaerobic digestion in a digester at elevated temperatures, during a period of in between 20 to 200 days in combination with an addition of further shredded medical or hospital waste such as bandages, clothes, linings,
wherein during the anaerobic digestion additional biodegrading enhancing supplements or agents are added into the digester in order to enhance the further acceleration of the nitril rubber degradation.

18. A method of producing an enhanced biodegradable nitril rubber glove, comprising the steps of:
forming around a glove former a first inner layer of a nitril rubber, wherein the nitril compound part comprise one of a pure NBR, a pure XNBR, a NBR, a XNBR compound or any combination thereof mixed with up to one of 20% weight percentage, 40% weight percentage, the first inner layer further comprising an amount of a biodegradable agent with one of proportions lower than or equal to 2 parts per 100 dry parts nitril rubber, between 1 and 2 parts per 100 dry parts nitril rubber; and
forming of an outer layer of a nitril rubber, wherein a nitril compound part comprise one of a pure NBR, a pure XNBR, a NBR, XNBR compound mixed with up to one of 20% weight percentage, 40% weight percentage, the outer layer further comprising an amount of biodegradable agent larger than one of 2 parts per 100 dry parts nitril rubber, 3 parts per 100 dry parts nitril rubber, 4 parts per 100 dry parts nitril rubber.

19. The method as according to claim 18, wherein after the forming of the first layer, and before the forming of the outer layer, an additional intermediate nitril rubber layer is formed on the glove former, the additional intermediate layer also comprising at least one of a biodegradable agent and a biobased filler material mixed inside the nitril rubber composition.

20. The method according to claim 18, wherein a method of biodegrading the nitril rubber glove comprising the steps of:
shredding the nitril rubber globe after use into smaller particles;
digesting the shredded small particles of the rubber globe by an anaerobic digestion in a digester at elevated temperatures, during a period of in between 20 to 200 days in combination with an addition of further shredded medical or hospital waste such as bandages, clothes, linings,
wherein during the anaerobic digestion additional biodegrading enhancing supplements or agents are added into the digester in order to enhance the further acceleration of the nitril rubber degradation.
